SEQUENCE LISTING (10 sequences)

SEQ ID NO: 1 - protein, 16 aa, Artificial sequence
  Synthetic peptide
VVLHPNYSQVDIGLIK

SEQ ID NO: 2 - protein, 32 aa, Artificial sequence
  Synthetic peptide
ADTHDEILEGLNFNLTEIPEAQIHEGFQELLR

SEQ ID NO: 3 - protein, 16 aa, Artificial sequence
  Synthetic peptide
EHEGAIYPDNTTDFQR

SEQ ID NO: 4 - protein, 18 aa, Artificial sequence
  Synthetic peptide
VDKDLQSLEDILHQVENK

SEQ ID NO: 5 - protein, 19 aa, Artificial sequence
  Synthetic peptide
GTAGNALMDGASQLMGENR

SEQ ID NO: 6 - protein, 24 aa, Artificial sequence
  Synthetic peptide
MVSHHNLTTGATLINEQWLLTTAK

SEQ ID NO: 7 - protein, 22 aa, Artificial sequence
  Synthetic peptide
QQQHLFGSSNVTDCSGNFCLFR

SEQ ID NO: 8 - protein, 16 aa, Artificial sequence
  Synthetic peptide
SVQEIQATFFYFTPNK

SEQ ID NO: 9 - protein, 13 aa, Artificial sequence
  Synthetic peptide
SWPAVGNCSSALR

SEQ ID NO: 10 - protein, 23 aa, Artificial sequence
  Synthetic peptide
SVQEIQATFFYFTPNKTEDIFLR

(19) United States
(12) Patent Application Publication
(10) Pub. No.: US 2022/0252616 A1
(43) Pub. Date: Aug. 11, 2022

(54) OPTIMIZED FRAGMENTATION FOR QUANTITATIVE ANALYSIS OF FUCOSYLATED N-GLYCOPROTEINS BY LC-MS-MRM

(71) Applicant: Georgetown University, Washington, DC (US)

(72) Inventors: Radoslav GOLDMAN, Reston, VA (US); Miloslav SANDA, Arlington, VA (US)

(21) Appl. No.: 17/618,802

(22) PCT Filed: Jun. 11, 2020
(86) PCT No.: PCT/US2020/037254

§ 371 Date: Dec. 13, 2021

Related U.S. Application Data
(60) Provisional application No. 62/861,709, filed on Jun. 14, 2019.

(51) Int. Cl. G01N 33/68

(52) U.S. Cl. CPC G01N 33/6893; G01N 33/6848; G01N 2800/085; G01N 2440/38; G01N 2333/75; G01N 33/6842

(57) ABSTRACT

Provided is a sensitive and specific LC-MS-MRM quantification method that distinguishes outer-arm and core fucosylated configurations of N-glycopeptides. Advantage is taken of limited fragmentation of the glycopeptides at low collision energy (collision-induced dissociation) CID to produce linkage-specific Y-ions. These ions are selected as multiple reaction monitoring (MRM) transitions for the quantification of the outer-arm and total fucosylation of 23 glycoforms of 9 glycopeptides in 7 plasma proteins. The method permits quantification of the glycoforms directly in plasma or serum without fractionation of samples or glycopeptide enrichment. A pilot study of fucosylation in liver cirrhosis of hepatitis C vims (HCV) and non-alcoholic steatohepatitis (NASH) etiologies demonstrated that liver cirrhosis is consistently associated with increased outer-arm fucosylation of a majority of the analyzed proteins. The outer-arm fucosyaltion of the A2G2F1 glycoform of the VDKDLQSLEDILHQVENK peptide of fibrinogen was found to increase more than 10-fold in the cirrhosis patients compared to healthy controls.

### RELATED APPLICATIONS

This application claims benefit of U.S. Provisional Patent Application No. 62/861,709, filed Jun. 14, 2019, the entire contents of which are incorporated herein by reference.

### GOVERNMENT SUPPORT

This invention was made with government support under OD023557, CA230692, and CA135069 awarded by the National Institutes of Health, and under CA51008 awarded by the National Cancer Institute. The government has certain rights in the invention.

### SEQUENCE LISTING

The instant application contains a Sequence Listing which has been submitted electronically in ASCII format and is hereby incorporated by reference in its entirety. Said ASCII copy, created on May 22, 2020, is named 706617_GUS-026PC_ST25.txt and is 3,132 bytes in size.

### BACKGROUND OF THE INVENTION

N-glycosylation of proteins is one of the most common posttranlational modifications. Alteration of N-glycans has been observed in various diseases and, specifically, fucosylation of the N-glycoproteins has been frequently associated with the diseases of the liver. Jia, L. et al. (2018) Front. Oncol. 8: 565; Miyoshi, E. et al. (2012) Biomolecules 2: 34; Morelle, W. et al. (2006) Glycobiology 16: 281; Comunale, M. A. et al. (2006) J. Proteome Res. 5: 308; Yuan, W. et al. (2015) J. Proteomics 116: 24; Sanda, M. et al. (2016) Anal. Bioanal. Chem. 409: 619; Wang, M. et al. (2017) Cancer Epidemiol. Biomarkers Prev. 26: 795. In human tissues, fucosylation of N-glycans takes place on the innermost GlcNAc (core fucosylation) by α-1,6 linkage, or on the outer arms by an α-1,2 linkage to a galactose (often terminal), or α-1,3 or α-1,4 linkage to the mostly subterminal GlcNAc based on the activity of specific fucosyltranserases. Staudacher, E. et al. (1999) Biochim. Biophys. Acta 1473: 216. The linkage of fucose, especially the core versus outer arm fucosylation, is an important determinant of the N-glycoprotein interactions and activities. Several published studies quantified core fucosylation in the context of liver disease and used either lectin affinity or endoglycosidase F (or H) digestion to achieve the quantification of core fucose. Cao, Q. et al. (2017) Methods Mol. Biol. 1619:127; Ma, J. et al. (2018) J. Proteomics DOI: 10.1016/j.prot.2018.02.003. However, a site- and linkage-specific quantification of the core and outer arm glycoforms was not achieved to our knowledge because of the technical challenges of these mass spectrometric assays.

Multiple reaction monitoring (MRM) workflows are widely used in quantitative proteomics due to their high sensitivity and specificity. In the recent years, this technique has been successfully employed for the quantification of glycopeptides. Yuan, W. et al. (2015) J. Proteomics 116: 24; Sanda, M. (2013) Mol. Cell Proteomics 12: 1294; Song, E. et al. (2012) Rapid Commun. Mass Spectrom. 26: 1941; Yang, N. et al. (2016) Anal. Chem. 88: 7091. Mass spectrometric quantification of glycopeptides is challenging because of the subtoichiometric representation of the microheterogeneous glycopeptides and their somewhat lower ionization efficiency; the reported workflows typically measure the low mass oxonium ions, such as m/z 204.1 (HexNAc), 366.1 (HexHexNAc), 138.1 (HexNAc-2H₂O—CH₂O), and 274.1 (Neu5Ac—H₂O). Goldman, R. et al. (2015) Proteomics Clin. Appl. 9: 17; Zhu, R. et al. (2017) Methods Mol. Biol. 1598: 213. The oxonium ions are the major fragment for all N-glycopeptides under collision-induced dissociation (CID) condition typical for peptide analysis; these ions have high sensitivity but low specificity and cannot distinguish between structural isomers. Therefore, analysis of isolated proteins, simple mixtures, or glycopeptide enrichment is typically used to reduce the sample complexicity. We have shown that Y-ions carrying the peptide backbone become the major fragments of the N-glycopeptides when the collision energy (CE) is lowered to approximately 50% of the optimal CE for peptide fragmentation. Sanda, M. et al. (2016) Anal. Bioanal. Chem. 409: 619; Yuan, W. et al. (2018) J. Proteome Res. 17: 2755. These Y-ions not only provide improved sensitivity (signal to noise) and specificity in complex samples but they can also yield characteristic fragments for specific structural features like core or outer arm fucosylated glycoforms. Yuan, W. et al. (2018) J. Proteome Res. 17: 2755; Ács, A. et al. (2018) Anal. Chem. 90: 12776. Therefore, a need still exists for an MRM workflow using optimized soft fragmentation yielding Y-ions as transitions for the study the fucosylation of major glycoproteins directly in plasma samples without prior enrichment of proteins or the glycopeptides.

### SUMMARY OF THE INVENTION

An aspect of the invention is a sensitive and selective method for the quantification of linkage specific fucosylation of glycoforms of plasma proteins without prior enrichment of proteins or the glycopeptides. The method comprises electing optimized soft fragments (Y-ions) instead of the commonly used oxonium ions as multiple reaction monitoring (MRM) transitions, thereby providing improved the sensitivity and specificity of quantification.

### BRIEF DESCRIPTION OF THE DRAWINGS

FIG. 1 is three graphs depicting optimized fragmentation selected for the quantification of changes in the fucosylation of the VVLHPNYSQVDIGLIK (SEQ ID NO: 1) peptide of haptoglobin. FIG. 1A depicts MSMS spectrum of A3G3F1 glycoform at low CE. FIG. 1B depicts MSMS spectrum of A3G3 glycoform at low CE. FIG. 1C depicts extracted ion chromatograms (XICs) of the selected soft fragments of A3G3 (orange), outer arm fucosylation (pink) and total fucosylation (green) of A3G3F1.

FIG. 2 is three graphs depicting impact of resolution at Q3 on the sensitivity and specificity of the measurement of A3G3F1 glycoform of the EHEGAIYPDNTTDFQR (SEQ ID NO: 3) peptide of ceruloplasmin. FIG. 2A depicts Q3 at Unit resolution. FIG. 2B depicts Q3 at Low resolution. FIG. 2C depicts Q3 at Open resolution. Red: transitions for outer arm fucosylation; Blue: transitions for total fucosylation.

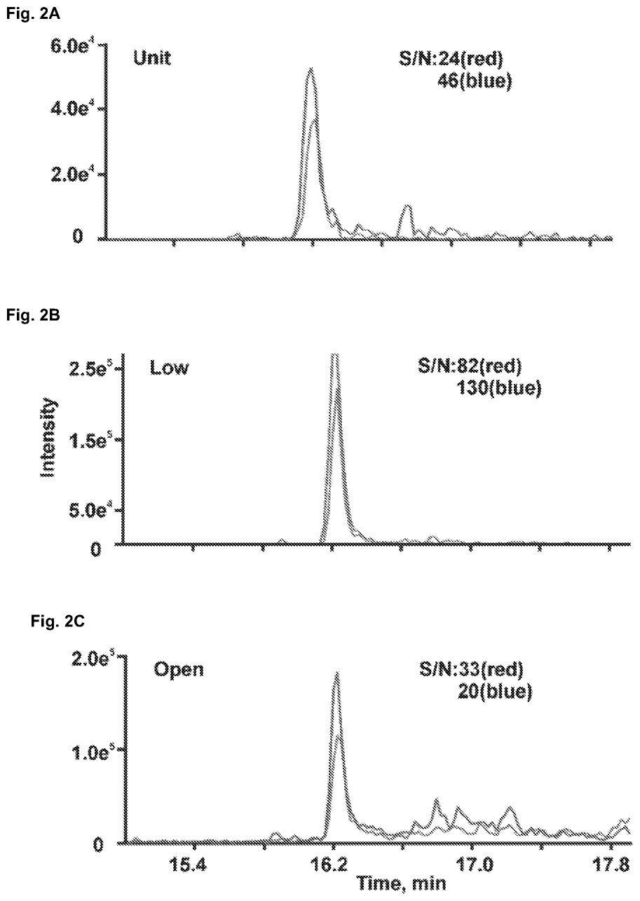

FIG. 3 is four graphs depicting XIC of the Y-ion and oxonium ion transitions of A3G3F1 and A3G3F2 glycoforms of the ADTHDEILEGLNFNLTEIPEAQIHEGFQELLR (SEQ ID NO: 2) peptide of antitrypsin under optimized CE conditions. FIG. 3A depicts Y-ion transition of A3G3F1 glycoform. FIG. 3B depicts Y-ion transition of A3G3F2 glycoform. FIG. 3C depicts oxonium ion transition of A3G3F1 glycoform. FIG. 3D depicts oxonium ion transition of A3G3F2 glycoform. Pink traces: transitions for outer arm fucosylation; Blue traces: transitions for total fucosylation; Red traces: M⁵⁺→204.1; Green traces: M⁵⁺→366.1.

FIG. 4 is a graph depicting outer-arm fucosylation of the A2G2F1 glycoform of the VDKDLQSLEDILHQVENK (SEQ ID NO: 4) peptide of fibrinogen in healthy control (n=5) and cirrhotic patients of the HCV (n=5) and NASH (n=5) etiologies.

FIG. 5A is three graphs depicting impact of resolution setting at Q3 on the sensitivity and specificity of total fucosylation soft fragments of A3G3F1 (in ADTHDEILEGLNFNLTEIPEAQIHEGFQELLR (SEQ ID NO: 2).

FIG. 5B is three graphs depicting impact of resolution setting at Q3 on the sensitivity and specificity of total fucosylation soft fragments of A3G3F2 in ADTHDEILEGLNFNLTEIPEAQIHEGFQELLR (SEQ ID NO: 2).

### DETAILED DESCRIPTION OF THE INVENTION

An aspect of the invention is a sensitive and selective method for the quantification of linkage specific fucosylation of glycoforms of plasma proteins without prior enrichment of proteins or the glycopeptides. The method comprises electing optimized soft fragments (Y-ions) instead of the commonly used oxonium ions as multiple reaction monitoring (MRM) transitions, thereby improving the sensitivity and specificity of quantification.

### Examples

### Materials and Methods

### Chemicals and Reagents

Dithiothreitol (DTT), acetonitrile and water in LC-MS grade were obtained from ThermoFisher Scientific (Waltham, Mass.). Iodoacetamide (IAA) was from MP Biomedicals (Santa Ana, Calif.). Trypsin Gold (V5280) and ProteaseMax (V2071) were from Promega (Madison, Wis.). α2-3,6,8,9 Neuraminidase A (P0722) and digestion buffers were from New England Biolabs (Ipswich, Mass.). All other chemicals were obtained from Sigma-Aldrich (St. Louis, Mo.).

### Study Population

All the participants were recruited under protocols approved by the Georgetown University Institutional Review Board. Blood samples were collected using BD vacutainer serum collection tube or EDTA Vacutainer tubes (BD Diagnostics, Franklin Lakes, N.J.) and the samples were processed within 6 h of the blood draw according to the manufacturer's protocol. Both plasma and serum samples were aliquoted and stored at −80° C. until use. Participants were further grouped into healthy controls (n=5), cirrhotic patients of hepatitis C virus (HCV, n=5), and non-alcoholic steatohepatitis (NASH, n=5) etiologies. The three groups were age-matched. HCV and NASH groups had comparable degree of liver damage as measured by model for end-stage liver disease (MELD) score (HCV 14.8 vs NASH 15.4).

### Tryptic Digest and Exoglycosidase Treatment

Plasma or serum was diluted in 50 mM ammonium bicarbonate buffer, reduced with 5 mM DTT, and alkylated with 15 mM Iodoacetamide which was quenched by 5 mM DTT. The samples were digested with Trypsin Gold in a Barocycler NEP 2320 (Pressure BioScience, Medford, Mass.) for one hour. ProteaseMax surfactant (0.03%) was added to the samples to improve the efficiency of digestion. Tryptic digests were then deactivated at 99° C. for 10 min and further treated with α2-3,6,8,9 Neuraminidase A in GlycoBuffer 1 (50 mM sodium acetate, 5 mM CaCl₂), pH 5.5) according to manufacturer's instructions. The Neuraminidase A treatment was conducted overnight at 37° C. At the end of digestion, Neuraminidase A was heat deactivated at 99° C. for 10 min. The digests were frozen at −80° C. for 30 min before centrifugation at 16,000 g for 10 min to facilitate the removal of the surfactant. The digests were subjected to liquid chromatography-tandem mass spectrometry (LC-MS/MS) analysis in randomized order without further processing to minimize sampling bias.

### Glycopeptide Quantification by LC-MS-MRM

Initial analysis of glycopeptides was carried out on an Orbitrap Fusion Lumos connected to a Dionex 3500 RSLC-nano-LC (Thermo Scientific) in a data-dependent mode in order to obtain the fragmentation information of the glycopeptides. Peptide and glycopeptide separation was achieved on a 150 mm×75 am C18 pepmap column by a 5 min trapping/washing step using 99% solvent A (2% acetonitrile containing 0.1% formic acid) at 10 μL/min followed by a 90 min acetonitrile gradient at a flow rate of 0.3 μL/min: 0-3 min 2% B, 3-5 min from 2% to 10% solvent B (0.1% formic acid in acetonitrile); 5-60 min from 10% to 45% solvent B; 60-65 from 35% to 98% solvent B; 65-70 min at 98% solvent B, 70.1-90 min equilibration by 2% solvent B. The electrospray ionization voltage was set to 2.3 kV, and the capillary temperature was set to 275° C. MS1 scans were performed over m/z 400-1800 with the wide quadrupole isolation on at a resolution of 120,000 (m/z 200), RF Lens was set to 40%, intensity threshold for MS2 was set to 2.0e4, selected precursors for MS2 were with charge state 2-7, Dynamic exclusion was set for 30s. Data-dependent higher energy collisional dissociation (HCD) tandem mass spectra were collected with a resolution of 15,000 in the Orbitrap with fixed first mass 110 and normalized collision energy 25%.

Quantitative analyses were performed in high mass positive ion mode on a 6500 Q-trap mass analyzer (AB Sciex, Framingham, Mass.) coupled with a nanoAcquity chromatographic system (Waters Associates, Milford, Mass.) consisting of a UPLC 2G Symmetry C18 TRAP column (5 μm, 180 μm×20 mm) and a BEH C18 300 capillary analytical column (1.7 μm particles, 75 μm×150 mm). Separation of the analytes was achieved by a 2 min trapping/washing step using 100% solvent A (2% acetonitrile containing 0.1% formic acid) at 15 μl/min followed by a 35 min acetonitrile gradient at a flow rate of 0.4 μl/min: 1 min from 1% to 5% solvent B (0.1% formic acid in acetonitrile) and 34 min from 5% to 50% solvent B. A cleaning gradient at a flow rate of 0.4 μL/min (10 min from 1% to 99% solvent B followed by a 10 min of isocratic run at 99% solvent B) with a 5 min sample trapping/washing step using 99% solvent B at 15 μl/min followed immediately after the peptide and glycopeptide separation in order to remove the nonpolar residues of ProteaseMax. Multiple reaction monitoring (MRM) mode was used for the quantification of glycopeptides with ion spray voltage set at 2300 V, curtain gas 11, ion source gas 1 30, and the interface heater temperature 180° C. Entrance potential (EP), collision cell exit potential (CXP), and declustering potential (DP) were set at 10 V, 13 V, and 75 V, respectively. Q1/Q3 were set at unit resolution. The plasma digest was analyzed on a 6600 TripleToF mass analyzer (AB Sciex, Framingham, Mass.) coupled with the nanoAcquity chromatographic system operated under the same chromatographic conditions described above. The MRM transitions for the glycoforms monitored in our experiments are listed in Table 1; oxonium ion m/z 512 was added to the transition list to monitor outer arm fucosylation. Collision energy (CE) for each MRM transition was optimized by a 5 V step optimization followed by a 1 V step fine tuning. Instrument control and data acquisition were performed by AB Sciex Analyst software (version1.7.1).

### Data Processing and Statistical Analysis

LC-MS-MRM data was processed by Multi Quant 2.0 software (AB Sciex) with manual confirmation of peak assignment. Peak areas were used for glycopeptide quantification and data normalization. The details of the MSMS transitions used for the quantification of each glycoform are listed in Table 1. Relative intensity of the outer-arm or total fucosylation was calculated by normalizing the respective peak area to the corresponding non-fucosylated glycoform.

### Results and Discussion

### Low CE (Soft) MRM Method Development

### Selection of MRM Transitions

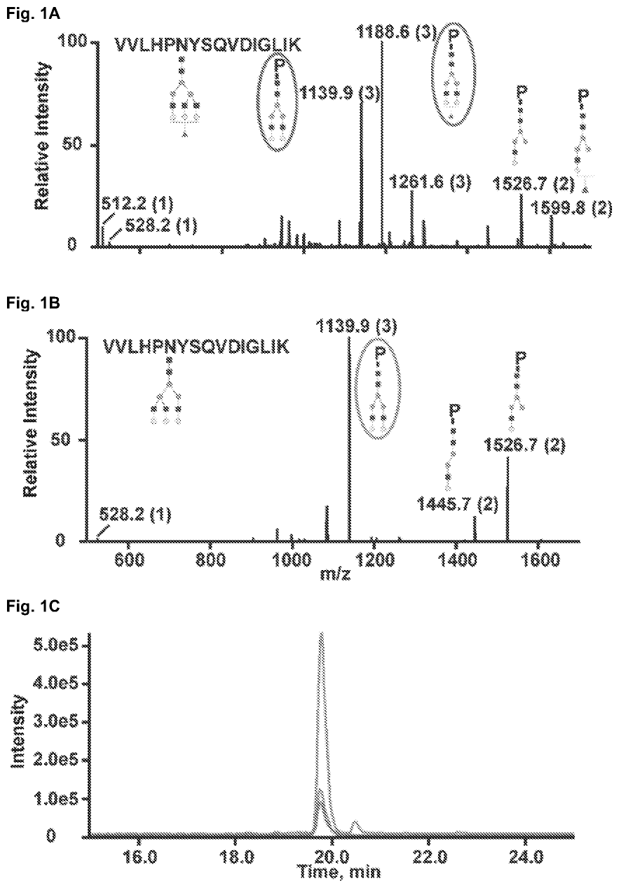

In our previous study using a data-independent method (Sanda, M. et al. (2016) Anal. Bioanal. Chem. 409: 619), we found a greater than 1.5-fold increase in the fucosylation of 125 glycoforms of 25 tryptic glycopeptides derived from 10 plasma proteins in cirrhotic patients compared to healthy controls. Both core- and outer arm-fucosylated glycoforms are elevated in the serum of the cirrhotic patients. Based on those results, we selected 23 glycoforms of 9 tryptic glycopeptides derived from 7 proteins (Table 1) to develop the MRM workflow for linkage specific quantitative fucosylation study in plasma. The rationale for the selection of the MRM transitions is documented in FIG. 1. The figure shows that, at the optimal CE, Y-ions become the major fragments for quantification of the A3G3 and A3G3F1 glycoforms of VVLHPNYSQVDIGLIK (SEQ ID NO: 1) peptide of haptoglobin. The MSMS spectrum of A3G3F1 glycoform (FIG. 1A) shows that both core- and outer arm-fucosylation contributes to the formation of the Y-ion m/z 1188.6. Therefore, this fragment is chosen to represent total fucosylation. On the other hand, Y-ion m/z 1139.9 resulting from the loss of a fucosylated GlcNAc-Gal arm of the N-glycan is the characteristic fragment of an outer arm fucosylated glycoform. This fragment stands for outer arm fucosylation. In addition to the soft fragments, the oxonium ion at m/z 512.2 is monitored to confirm the outer arm fucosylation. Because of the lack of isotopically labeled standards of the glycopeptides, we normalize the integrated peak area of the total or outer-arm fucosylated transitions to its corresponding non-fucosylated glycoform for the purpose of quantification; in the case of the VVLHPNYSQVDIGLIK (SEQ ID NO: 1) peptide of haptoglobin we normalize to the A3G3 glycoform (FIG. 1B). Y ion m/z 1139.9, resulting from the loss of one GlcNAc-Gal arm (dissociation of the Man-GlcNAc bond), is chosen to quantify the A3G3 glycoform. The XIC corresponding to these MRM transitions is shown in FIG. 1C and the MRM transitions of all glycoforms targeted in this study are chosen based on the same principle and listed in Table 1. Transitions for sialylated glycoforms (A2G2S1 and A2G2S2) of VVLHPNYSQVDIGLIK (SEQ ID NO: 1) peptide of haptoglobin are added to monitor the completion of Neuraminidase A digestion. CEs for each MRM transition were optimized to achieve maximum signal intensity. The glycoforms and their retention times were verified by running the same plasma digest on a 6600 TripleToF mass analyzer coupled with the NanoAcquity chromatographic system using the same chromatographic conditions.

### Impact of Resolution in Q3 on Sensitivity and Specificity

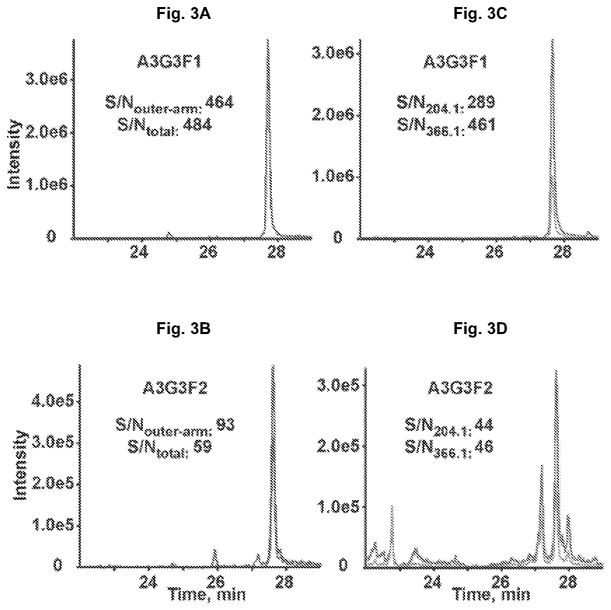

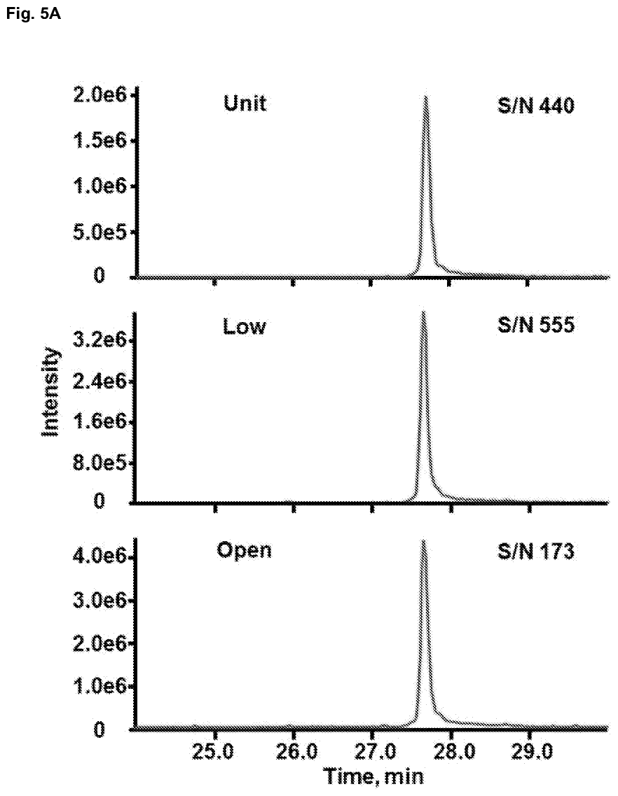

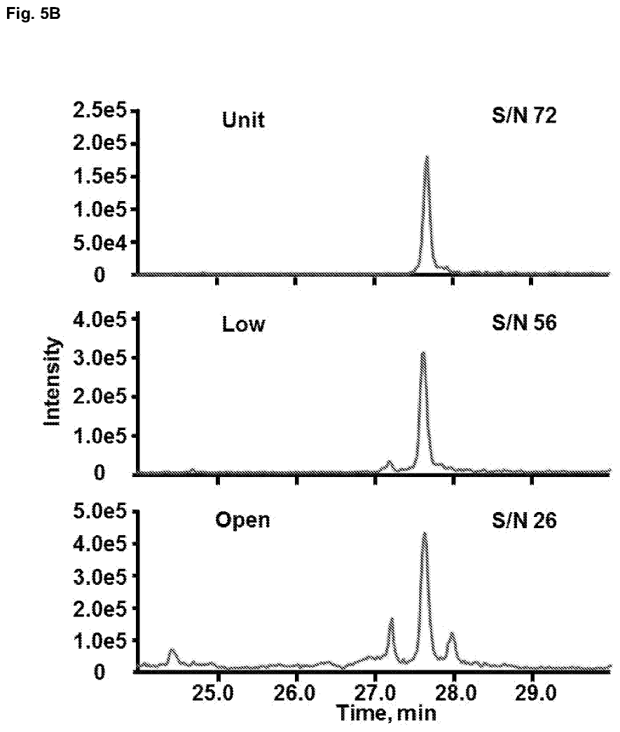

The settings of resolution for the Q1 and Q3 can affect both sensitivity and specificity; lower resolution results in higher sensitivity but lower specificity. In this study, Q1 was set at Unit resolution to increase the specificity of precursor ion selection. The sensitivity and specificity at different resolution settings (Unit, Low, and Open) at Q3 were compared. The intensity of analyte peaks increased when the resolution was lowered from Unit to Low while specificity remains comparable (FIG. 5). The intensities of the high and moderate abundant analytes, such as the A3G3F1 and A3G3F2 in ADTHDEILEGLNFNLTEIPEAQIHEGFQELLR (SEQ ID NO: 2) of antitrypsin (FIG. 5A and FIG. 5B), increased more than 1.5 fold compared to the Unit resolution. At the same time, the background remains low so that the S/N ratios of these peaks improve compared to the Unit resolution. At Open resolution, the background signal decreased the S/N ratio and the interference peaks reduced the specificity of detection (FIG. 5B). For the low abundant analytes, such as the A3G3F1 glycoform of EHEGAIYPDNTTDFQR (SEQ ID NO: 3) peptide of ceruloplasmin (FIG. 3), low resolution leads to a 4 fold increase of the intensity of the targeted peaks but a minimal increase in the background noise. The resulting S/N ratios of both outer-arm and total fucosylation transitions increased over 3 fold compared to the unit resolution for this analyte. We therefore chose the Low resolution setting of Q3 for the quantification of the analytes in this study.

### Comparison of Y- and Oxonium Ion Transitions

More than half of the Y-ions in this study have an m/z greater than 1250, beyond the upper limit of the low mass mode on the Qtrap 6500. We therefore developed the quantification method in the high mass mode. In general, the high mass mode is about 5 times less sensitive compared to the low mass mode. However, the specificity of the high mass fragments outweighs the loss of sensitivity in the high mass mode on this mass spectrometer. We compared the specificity and sensitivity of Y-ion transitions in high mass mode to the oxonium ion transitions in low mass mode. When the Y-ions of A3G3F1 and A3G3F2 of ADTHDEILEGLNFNLTEIPEAQIHEGFQELLR (SEQ ID NO: 2) of antitrypsin were compared to their corresponding oxonium ion transitions (FIG. 3), the Y-ions provided up to 2-times higher S/N ratio. The less specific oxonium ion transitions produce interference peaks to a greater degree as shown on the case of A3G3F2 transitions of this glycopeptide (FIG. 3D) compared to the Y-ions (FIG. 3B). Similar results were observed for most of the monitored glycoforms which suggests that the use of Y-ions generated by soft fragmentation is advantageous even with the lower sensitivity of the high mass mode on this mass spectrometer.

### Reproducibility

A quality control (QC) sample was used to assess the reproducibility of the method. The QC plasma sample was analyzed repeatedly and relative quantification of all the targeted glycoforms was performed by normalizing the integrated peak area of total or outer-arm fucosylated transitions to their corresponding nonfucosylated glycoform (Table 2). The average RSD across the analytes is 11.6% and ranges from 1.5 to 23%. We also checked the influence of the protein load on the performance of the assays by measuring the RSDs of 0.75, 1, or 2 μg total plasma protein. Our results show that the average RSD of the measurements is 14% and that RSD of any measurement is below 22% except one glycoform of ceruloplasmin (Table 3). Thus, the optimized soft fragment MRM method achieves sensitive and reliable quantification of the glycoforms in the tryptic digest of plasma without any fractionation or enrichment. At the same time, the method allows quantitative analysis of specific fucosylation structures by separate quantification of the core fucosylation linkage.

Quantification of the Fucosylated Glycoforms in the Plasma of Patients with Liver Cirrhosis

The energy-optimized MRM workflow was used to quantify the linkage specific fucosyaltion change of 12 glycoforms in 7 plasma proteins in cirrohotic patients of HCV (n=5) or NASH (n=5) etiologies compared to disease free controls (n=5). Except for the A2G2F1 glycoform of the SWPAVGNCSSALR (SEQ ID NO: 9) peptide of hemopexin (HCV and NASH etiology) and the A3G3F2 glycoform of ADTHDEILEGLNFNLTEIPEAQIHEGFQELLR (SEQ ID NO: 2) peptide of antitrypsin (NASH etiology), both outer-arm and total fucosylation increased significantly in the HCV and NASH patients compared to the healthy controls (Table 4, >1.6-fold, p≤0.05). We did not find significant differences between the HCV and NASH patients, in line with the notion that cirrhotic changes are the major contributor to the observed differences irrespective of the etiology. This is in agreement with the results from our lab and other labs. Yuan, W. et al. (2015) J. Proteomics 116: 24; Sanda, M. et al. (2016) Anal. Bioanal. Chem. 409: 619; Yuan, W. et al. (2018) J. Proteome Res. 17: 2755; Benicky, J. et al. (2014) Anal. Chem. 86: 10716; Comunale, M. A. et al. (2006) J. Proteome. Res. 5: 308; Comunale, M. A. et al. (2010) PLoS One 5: e12419; Zhu, J. et al. (2014) J. Proteome Res. 13: 2986. The observed fold changes in fucosylation of the outer-arm are similar to those of total fucosylation, suggesting that core fucosylation represents a minor contribution to the observed changes. The outer-arm fucosylation is likely more sensitive to the liver damage in line with our previous studies. Yuan, W. et al. (2018) J. Proteome Res. 17: 2755.

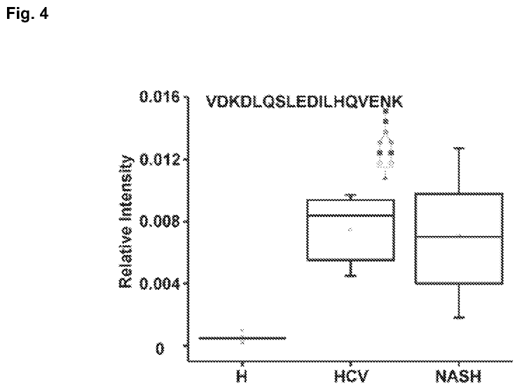

Interestingly, we observed the most significant fold change (>10-fold, p≤0.01) on the fucosylated outer-arm of the A2G2F1 glycoform of the VDKDLQSLEDILHQVENK (SEQ ID NO:4) peptide of fibrinogen γ-chain (Asn52) (FIG. 4). It was reported that sialylation of the N-glycans of fibrionogen affects its clotting behavior (Dang, C. V. et al. (1989) J. Biol. Chem. 264: 15104) and increased fucosylation and sialylation of fibrinogen has been reported in cirrhotic patients with/out HCC (Nagel, T. et al. (2018) Anal. Bioanal. Chem. 410: 7965) which is consistent with our findings. Moreover, our results suggest that outer-arm fucosylation of the A2G2F1 glycoform of this peptide of fibrinogen is more sensitive to the fibrotic changes of the liver than fucosylation of other glycoproteins. The Asn52 of fibrinogen is adjacent to a central hinge point located in a non-helical segment of the γ chain, where the coiled-coils region of fibrinogen can bend around. Marsh, J. J. et al. (2013) Biochemistry 52: 5491. The coiled-coil region has been ascribed to the tensile deformation of fibrin fibers. Weisel, J. W. et al. (2017) Subcell. Biochem. 82: 405; Zhmurov, A. et al. (2011) Structure. 19: 1615; Zhmurov, A. et al. (2012) J. Am. Chem. Soc. 134: 20396. Fibrin is formed after enzymatic release of fibrinopeptides from fibrinogen and further polymerized to form fibrin clot. Reports on computational analysis of fibrinogen suggest that the carbohydrate moieties on Asn52 of the γ-chain and Asn364 of the β-chain affect the B-b knob-hole interactions in fibrin (Weisel, J. W. et al. (2017) Subcell. Biochem. 82: 405), and this knob-hole interaction has been proposed to affect the susceptibility of clots to proteolytic digestion. Doolittle, R. F. et al. (2006) Biochemistry 45: 2657. We also noticed an increase in both the outer-arm (>6-fold, P≤0.01) and total (>4-fold, P≤0.01) fucosylation of the A2G2F1 glycoform of the GTAGNALMDGASQLMGENR (SEQ ID NO: 5) (Asn364) β-chain of fibrinogen (Table 4) in the cirrhotic patients.

Serum is the commonly used blood sample obtained by the removal of the clotting factors and fibrinogen. Our results suggest that plasma which contains the fibrinogen may be the desired blood fraction for the quantification of the fibrotic liver disease. We fully acknowledge that this small proof of principle quantification study was not designed to test this hypothesis and needs to be expanded to justify the observation. Nonetheless, we evaluated whether the sample matrix (serum vs plasma) affects the quantitative results of analytes present in both serum and plasma. To this end, we analyzed paired serum and plasma of patients with cirrhosis of HCV etiology using our optimized workflow. Our results show that the fold changes measured in serum are consistent with the paired plasma samples and we did not detect any significant differences in those two sample sets (Table 5). This further confirms the reliability of our quantitative measurement and suggests that the fucosylation of fibrinogen is indeed more sensitive than the other proteins to the fibrotic changes of the liver in this small sample set.

### CONCLUSION

We successfully optimized a sensitive and selective method for the quantification of linkage specific fucosylation of 12 glycoforms of 7 plasma proteins. Selecting the optimized soft fragments (Y-ions) instead of the commonly used oxonium ions as MRM transitions improved the sensitivity and specificity of quantification. Our results document that the workflow can be applied to the quantification of the glycoforms of abundant proteins directly in plasma or serum without fractionation or glycopeptide enrichment. This minimizes artifacts of sample processing and simplifies the analytical protocol. The Y-ions also provide information on fucose linkage and allowed us to resolve partially the linkage isoforms of the fucosylated glycopeptides. This is a desired improvement allowing association of the activity of specific glycosyltransferases with biomarker discovery which results in an improved understanding of the biology of the diseases. We applied the workflow to a pilot study of fucosylation of N-glycoproteins in liver cirrhosis of the HCV and NASH etiologies. The results reveal that increased outer-arm fucosylation of majority of the proteins is consistently associated with the development of liver cirrhosis. We have found that the outer-arm fucosyaltion of the A2G2F1 glycoform of the VDKDLQSLEDILHQVEN (SEQ ID NO: 4) peptide of fibrinogen increases greater than 10 times in this small pilot study of cirrhosis in the HCV and NASH patients. In summary, we report a novel LC-MS-MRM workflow for the quantification of site- and linkage-specific fucosylation based on energy optimized fragmentation of the N-glycopeptides. The quantification was achieved directly in a tryptic digest of serum or plasma proteins without fractionation or glycopeptide enrichment.

LIST OF SEQUENCES

| | |
|---|---|
| VVLHPNYSQVDIGLIK | SEQ ID NO: 1 |
| ADTHDEILEGLNFNLTEIPEAQIHEGFQELLR | SEQ ID NO: 2 |
| EHEGAIYPDNTTDFQR | SEQ ID NO: 3 |
| VDKDLQSLEDILHQVENK | SEQ ID NO: 4 |
| GTAGNALMDGASQLMGENR | SEQ ID NO: 5 |
| MVSHHNLTTGATLINEQWLLTTAK | SEQ ID NO: 6 |
| QQQHLFGSSNVTDCSGNFCLFR | SEQ ID NO: 7 |
| SVQEIQATFFYFTPNK | SEQ ID NO: 8 |
| SWPAVGNCSSALR | SEQ ID NO: 9 |
| SVQEIQATFFYFTPNKTEDIFLR | SEQ ID NO: 10 |

TABLE 1
MRM transitions of the targeted glycoforms.

| | | | | | Loss | Loss of | | |
|---|---|---|---|---|---|---|---|---|
| | | | | | of F-arm | nonF-arm | | |
| | | | | Precursor | Soft | Soft | | |
| | C(Carbamidomethyl) | Glycan | Precursor | Charge | Fragment | Fragment | Oxonium | RT |
| Protein | Peptide | Composition | m/z | State | m/z | m/z | Ion | (min) |
| | | | | | 3+ | 3+ | | |
| HAPTOGLOBIN | M(O)VSHHNLTTGATLINEQW | A3G3 | 1171.5 | 4 | N/A | 1440.0 | N/A | 23.67 |
| | LLTTAK | A3G3F1 | 1208.0 | 4 | 1440.0 | 1488.7 | 512.2 | 23.64 |
| | VVLHPNYSQVDIGLIK | A2G2 | 855.2 | 4 | N/A | 1018.2 | N/A | 20.84 |
| | | A2G2F1 | 891.7 | 4 | 1018.2 | 1066.8 | 512.2 | 20.74 |
| | | A3G3 | 946.4 | 4 | N/A | 1139.9 | N/A | 20.65 |
| | | A3G3F1 | 983.0 | 4 | 1139.9 | 1188.6 | 512.2 | 20.61 |
| SEROTRANSFERRIN | QQQHLFGSNVTDCSGNFCLFR | A3G3 | 1126.5 | 4 | N/A | 1379.9 | N/A | 22.06 |
| | | A3G3F1 | 1163.0 | 4 | 1379.9 | 1428.6 | 512.2 | 21.99 |
| | | | | | 4+ | 4+ | | |
| ANTITRYPSIN | ADTHDEILEGLNFNLTEIPEAQ- | A3G3 | 1136.7 | 5 | N/A | 1329.4 | N/A | 29.45 |
| | IHEGFQELLR | A3G3F1 | 1165.9 | 5 | 1329.4 | 1365.9 | 512.2 | 29.41 |
| | | A3G3F2 | 1195.1 | 5 | 1365.9 | 1402.4 | 512.2 | 29.38 |
| | | | | | 3+ | 3+ | | |
| FIBRINOGEN | VDKDLQSLEDILHQVENK | A2G2 | 937.2 | 4 | N/A | 1127.5 | N/A | 23.98 |
| | | A2G2F1 | 973.7 | 4 | 1127.5 | 1176.2 | 512.2 | 23.93 |
| | GTAGNALMDGASQLMGENR | A2G2 | 879.6 | 4 | N/A | 1050.8 | N/A | |
| | | A2G2F1 | 916.1 | 4 | 1050.8 | 1099.5 | 512.2 | 21.22 |
| ALPHAl-ACID | SVQEIQATFFYFTPNK | A4G4 | 1069.0 | 4 | N/A | 1303.2 | N/A | 24.09 |
| GLYCOPROTEIN | | A4G4F1 | 1105.5 | 4 | 1303.2 | 1351.9 | 512.2 | |
| Ceruloplasmin | EHEGAIYPDNTTDFQR | A3G3 | 970.9 | 4 | N/A | 1172.5 | N/A | 16.62 |
| | | A3G3F1 | 1007.4 | 4 | 1172.5 | 1221.2 | 512.2 | 16.61 |
| | | | | | 2+ | 2+ | | |
| Hemopexin | SWPAVGNCSSALR | A2G2 | 1009.8 | 3 | N/A | 1331.6 | N/A | 17.62 |
| | | A2G2F1 | 1058.4 | 3 | 1331.6 | 1404.6 | 512.2 | 17.6 |
| | | A3G3 | 1131.5 | 3 | N/A | 1514.1 | N/A | 17.58 |
| | | A3G3F1 | 1180.2 | 3 | 1514.1 | 1587.2 | 512.2 | 17.57 |
| QC for | VVLHPNYSQVDIGLIK | A2G251 | 927.9 | 4 | 1526.7 | 1445.7 | N/A | 274.1 |
| Neuraminidase A | | A2G252 | 1000.7 | 4 | 1526.7 | 1344.2 | N/A | |
| HAPTOGLOBIN | | | | | | | | |

Loss of F-arm or nonF-arm soft fragment stands for the fragment resulting from the loss of a fucosylated or non-fucosylated GlcNAc-Gal arm of the N-glycan, respectively.
The oxonium ion m/z 512.2 transition was monitored for all fucosylated glycoforms.

TABLE 2
Reproducibility of the measurements expressed as RSD (%) of the
quantification (n = 5).

| | | | RSD(%) | |
|---|---|---|---|---|
| | | | outer | |
| protein | peptide | glycoform | arm | total |
| Haptoglobin | M(O)VSHHNILITGATUNEQWLLTTAK | A3G3F1 | 14.4 | 12.5 |
| | VVLHPWSQVDIGLIK | A2G2F1 | 13.8 | 6.1 |
| | | A3G3F1 | 15.1 | 10.0 |
| Serotransferrin | QQQHLFGSNVTDCSGNFOLFR | A3G3F1 | 20.4 | 21.2 |
| Antitrypsin | ADTHDEELEGINFNLTEEPEAQIHEGFQELLR | A3G3F1 | 12.2 | 11 8 |
| | | A3G3F2 | 21.5 | 23.0 |
| Fibrinogen | VDKDLQSLEDILHQVENK | A2G2F1 | 11 5 | NA |
| | GTAGNALMDGASQLMGENR | A2G2F1 | 13.1 | 14.6 |
| Alpha-acid | SVQEIQATFFYFTPNK | A4G4F1 | 4.6 | 0.9 |
| glycoprotein | | | | |
| Cerulopasmin | EHEGAIYPONTTDFOR | A3G3F1 | 8.8 | 7.8 |
| Hemopexin | SWPAVGNICSSALR | A2G2F1 | 4 1 | 1.5 |
| | | A3G3F1 | 11.3 | 6.5 |

TABLE 3
Effect of sample loading on the measurement

| | | | RSD(%) | |
|---|---|---|---|---|
| | | | outer | |
| protein | peptide | glycoform | arm | total |
| Haptoglobin | M(O)VSHHNLTTGATLINEQWLLTTAK | A3G3F1 | 18.7 | 16.0 |
| | VVLHPNYSQVDIGLIK | A2G2F1 | 3.2 | 3.9 |
| | | A3G3F1 | 22.0 | 18.8 |
| Serotransferrin | QQQHLFGSNVTDCSGNFCLFR | A3G3F1 | 1.3 | 8.7 |
| Antitrypsin | ADTHDEILEGLNFNLTEIPEAQIHEGF | A3G3F1 | 3.8 | 2.3 |
| | QELLR | A3G3F2 | 5.3 | 5.8 |
| Fibnnogen | VDKDLQSLEDILHQVENK | A2G2F1 | 11.1 | |
| | GTAGNALMDGASQLMGENR | A2G2F1 | 5.3 | 7.4 |
| Alpha-acid | SVQEIQATFFYFTPNK | A4G4F1 | 1.2 | 1.0 |
| glycoprotein | SVQEIQATFFYFTPNKTEDTIFLR | A4G4F1 | 7.5 | 2.3 |
| CeruloOasmin | EHEGAIYPDNTTDFQR | A3G3F1 | 28.6 | 15.1 |
| Hemopexin | SWPAVGNCSSALR | A2G2F1 | 13.1 | 8.6 |
| | | A3G3F1 | 9.8 | 14.8 |

RSD was calculated from the results of 0.75, 1 and 2 μg of total protein digest analyses

TABLE 4
Total and outer fucosylation fold changes in HCV and
NASH patients (data were compared to healthy control)

| | Fold | | Student | |
|---|---|---|---|---|
| | change/H | | t test (p)/H | |
| | HCV | NASH | HCV | NASH |
| M.O.VSHHNLTTGATLINEQWLLTTAK. | 2.9 | 3.1 | <0.01 | 0.01 |
| A3G3F1-outer | | | | |
| M.O.VSHHNLTTGATLINEQWLLTTAK. | 3.4 | 3.5 | <0.01 | 0.01 |
| A3G3F1-total | | | | |
| VVLHPNYSQVDIGLIK.A2G2F1-outer | 2.4 | 3.6 | <0.01 | 0.02 |
| VVLHPNYSQVDIGLIK.A2G2F1-total | 2.2 | 3.8 | <0.01 | 0.03 |
| VVLAPNYSQVDIGLIK.A3G3F1-outer | 4.8 | 5.4 | <0.01 | 0.02 |
| VVLAPNYSQVDIGLIK.A3G3F1-total | 4.8 | 6.0 | <0.01 | 0.02 |
| QQOHLFGSNVTDCSGNFCLFR.A3G3F1-outer | 2.1 | 2.5 | <0.01 | 0.05 |
| QQQHLFGSNVTDCSGNFCLFR.A3G3F1-total | 2.2 | 2.7 | <0.01 | 0.03 |
| ADTHDEILEGLNFNLTDPEAQIHEGFQELLR. | 2.5 | 2.9 | <0.01 | 0.05 |
| A3G3F1-outer | | | | |
| ADTHDEILEGLNFNLTDPEAQIHEGFQELLR. | 2.3 | 2.8 | <0.01 | 0.0 |
| A3G3F1-total | | | | |
| ADTHDEILEGLNFNLTDPEAQIHEGFQELLR. | 3.4 | 7.3 | <0.01 | 0.14 |
| A3G3F2-outer | | | | |
| ADTHDEILEGLNFNLTDPEAQIHEGFQELLR. | 3.2 | 6.9 | <0.01 | 0.15 |
| A3G3F2-total | | | | |
| VDKDSLEDILHQVENK.A2G2F1-outer | 14.1 | 13.3 | <0.01 | 0.01 |
| SVQEIQATFFYFTPNK.A4C4F1-outer | 4.6 | 5.8 | <0.01 | 0.01 |
| SVQEIQATFFYFTPNK.A4G4F1-total | 3.6 | 5.0 | <0.01 | 0.02 |
| EHEGAIVPDNTTDFQR.A3G3F1-outer | 1.9 | 2.3 | <0.01 | 0.05 |
| EHEGAIYPDNTTDFQR.A3G3F1-totai | 1.6 | 2.4 | 0.01 | 0.05 |
| SWPAVGNCSSALR.A2G2F1-outer | 1.3 | 1.6 | 0.10 | 0.09 |
| SWPAVGNCSSALR.A2G2F1-total | 1.1 | 1.8 | 0.76 | 0.12 |
| SWPAVGNCSSALR.A3G3F1-outer | 3.4 | 4.0 | <0.01 | 0.01 |
| SWPAVGNCSSALR.A3G3F1-total | 2.6 | 3.5 | <0.01 | <0.01 |

TABLE 5
Fold change of glycoforms measured in paired
plasma and serum samples of HCV patients.

| | Fold change/H | |
|---|---|---|
| glycoforms | plasma | serum |
| QQQHLFGSNVTDCSGNFCLFR.A3G3F1-outer | 2.1 | 2.4 |
| QQQHLFGSNVTDCSGNFCLFR.A3G3F1-total | 2.2 | 2.9 |
| ADTHDEILEGLNFNLTEIPEAQIHEGFQELLR. | 2.5 | 2.5 |
| A3G3F1-outer | | |
| ADTHDEILEGLNFNLTEIPEAQIHEGFQELLR. | 2.3 | 2.2 |
| A3G3F1-total | | |
| ADTHDEILEGLNENLTEIPEAQIHEGFQELLR. | 3.4 | 3.3 |
| A3G3F2-outer | | |
| ADTHDEILEGLNFNLTEIPEAQIHEGFQELLR. | 3.2 | 3.3 |
| A3G3F2-total | | |
| SVQEIQATFFYFTPNK.A4G4F1-outer | 4.6 | 4.9 |
| SVQEIQATFFYFTPNK.A4G4F1-total | 3.6 | 3.7 |
| EHEGAIYPDNTTDFQR.A3G3F1-outer | 1.9 | 1.6 |
| EHEGAIYPDNTTDFQR.A3G3F1-total | 1.6 | 1.3 |

1. A sensitive and selective method for the quantification of linkage specific fucosylation of glycoforms of plasma proteins without prior enrichment of proteins or glycopeptides, comprising electing optimized soft fragments (Y-ions) instead of 5 commonly used oxonium ions as multiple reaction monitoring (MRM) transitions, thereby improving sensitivity and specificity of quantification.

2. The method of claim 1, wherein the plasma proteins are selected from the group consisting of haptoglobin, serotransferrin, antitrypsin, fibrinogen, alpha-acid glycoprotein, ceruloplasmin, and hemopexin.

3. The method of claim 1, wherein the plasma protein is fibrinogen.

4. The method of claim 1, wherein the oxonium ions are selected from the group consisting of m/z 204.1 (HexNAc), m/z 366.1 (HexHexNAc), m/z 138.1 (HexNAc-2H₂O—CH₂O), and m/z 274.1 (Neu5Ac—H₂O).

5. The method of claim 1, wherein the plasma proteins are present in a sample of plasma or serum of a patient.

6. The method of claim 5, wherein the subject has liver cirrhosis.
